(19) United States
(12) Patent Application Publication
(10) Pub. No.: US 2007/0270742 A1
(43) Pub. Date: Nov. 22, 2007

(54) Kit for Introducing an Intragastric Implant, a cartridge for introducing Such an Implant, and a Corresponding Method of Manufacture

(75) Inventor: Richard Jean-Claude Guetty, Miribel (FR)

Correspondence Address: Thomas Kayden Horstemeyer & Risley, Suite 1750, 100 Galleria Parkway N W, Atlanta, GA, 30339-5948, US

(73) Assignee: Compagnie Europeenne D'Etude Et De Recherche De Dispositifs Pour L' Implantation Par Laparoscopie, Vienne (FR)

(21) Appl. No.: 10/552,905

(22) PCT Filed: Apr. 13, 2004
(86) PCT No.: PCT/FR04/00898

§ 371 Date: Jan. 25, 2007

(30) Foreign Application Priority Data
Apr. 16, 2003 (FR) 03/04794

(51) Int. Cl. A61M 29/02

(52) U.S. Cl. 604/103.05; 606/192

(57) ABSTRACT

A kit for introducing an intragastric implant. The kit comprises an intragastric implant for implanting in the stomach. The implant is expandable from a configuration for introduction into the stomach to a therapeutic configuration within the stomach. The kit further includes a cartridge for packaging said implant in the introduction configuration. The cartridge is provided with an opener member activatable by positive action enabling it to depart from a closed configuration. The cartridge includes locking means functionally connected to the opener member and capable on its own, without any external action on said locking means of holding the cartridge in the closed configuration. The intragastic implant may be used for treating obesity.

### TECHNICAL FIELD

The present invention relates to the general technical field of surgical implants for implanting inside a patient's body, in a cavity that is natural (e.g. defined by an organ forming a pouch or a duct), or that is artificial (provided surgically).

The invention relates more specifically but not exclusively to the technical field or artificial devices for treating obesity, in particular morbid obesity, and more particularly devices seeking to reduce, artificially, the volume of the gastric cavity in order to create in the patient a sensation of being sated rapidly.

The present invention relates more particularly to a kit for introducing a surgical implant into a cavity in the body of a patient, the kit comprising:

a surgical implant for implanting in said cavity, said implant being expandable from a configuration for introduction into the cavity to a therapeutic configuration within the cavity; and

a cartridge for packaging said implant in the introduction configuration, said cartridge being provided with an opener member activatable by positive action enabling the cartridge to pass from a closed configuration in which it confines the implant in its introduction configuration, to an open configuration in which it allows said implant to expand.

The present invention also relates to a cartridge for introducing a surgical implant into a cavity within the body of a patient, said implant being designed to be implanted in said cavity and being expandable from a configuration for introduction into the cavity to a therapeutic configuration within the cavity, said cartridge being designed to package said implant in its introduction configuration and being provided with an opener member that is activatable by positive action enabling the cartridge to pass from a closed configuration in which it confines the implant in its introduction configuration, to an open configuration in which it allows said implant to expand.

Furthermore, the invention relates to a method of manufacturing a kit for introducing into the stomach of a patient an intragastric implant for treating obesity, the method comprising:

supplying or making an intragastric implant for implanting in the stomach in order to reduce its volume, said implant being expandable from a configuration for introduction into the stomach to a therapeutic configuration within the stomach;

supplying or making a cartridge for packaging said implant in the introduction configuration; and

providing said cartridge with an opener member activatable by positive action enabling the cartridge to pass from a closed configuration in which it is suitable for confining the implant in its introduction configuration, to an open configuration in which it is suitable for allowing said implant to expand.

Finally, the present invention relates to a novel use of a chain stitch.

### PRIOR ART

In order to treat patients suffering from obesity, in particular those presenting a ratio of weight over height that does not require recourse to invasive surgical devices and methods that are expensive and traumatic, such as surgically implanting a gastric ring, and also for treating patients having excess weight that is itself considered as constituting a risk in the event of surgery, it is known to implant a foreign body directly in the stomach of the patient, the foreign body being of sufficient volume to reduce the space available for food, and also to reduce its transit speed.

Such foreign bodies are implanted via the mouth, generally under endoscopic control, and they are usually in the form of so-called “intragastric” balloons formed by a flexible bag made of a biocompatible elastomer material and implanted directly in the stomach of the patient.

The balloon presents an orifice having a valve installed therein, with these two elements forming connection means into which the surgeon introduces a connection member prior to implanting the balloon in its non-expanded state, the connection member generally being a catheter connected to a source of fluid (physiological liquid and/or gas) so as to make it possible subsequently to inflate the balloon while it is in the stomach.

The balloon is generally positioned within the stomach as follows:

while in its non-expanded configuration, the balloon is folded (e.g. rolled up or twisted) so as to present a shape that is generally oblong;

the balloon when folded in this way is placed inside a cover serving simultaneously to protect the balloon against possible external attack, to keep the balloon in its folded configuration, and also to act as a pod around the balloon to make it easier to introduce into the stomach via the natural path; and

the assembly constituted by the cover containing the folded balloon is introduced via the mouth and the esophagus into the stomach of the patient.

It is known to make the cover out of elastomer material and to provide weakness means such as slits. Thus, once the folded balloon has been introduced into the patient's stomach, the balloon is inflated via the above-mentioned catheter, thereby expanding the cover of elastomer material until it bursts, where bursting of the cover is made easier by the slits of weakness. The catheter and the cover are then withdrawn from the patient's body, leaving the balloon alone in the stomach.

The use of such a cover of pre-slit elastomer material nevertheless presents numerous drawbacks.

Firstly, putting the intragastric balloon into place via the natural path requires the assembly constituted by the sheath containing the balloon to present a section that is as small as possible in order to facilitate passage through the esophagus and limit patient discomfort.

Unfortunately, using a cover of elastomer material does not enable the folded balloon to be thoroughly compressed, but serves no more than to sheathe it, i.e. to fit over its shape and volume.

Thus, the use of an elastomer cover does not enable the volume of the balloon to be further reduced after folding, nor does it enable the diameter of the assembly formed by the cover and the balloon to be made uniform along its length.

Consequently, the use of an elastomer cover does not enable the assembly that is introduced into the esophagus to be made as compact as possible. This is particularly true when the intragastric balloon comprises two flexible bags, the first being smaller in volume than the second and being disposed inside the second, as described in patent application PCT/FR 02/04589 in the name of the Applicant.

U.S. Pat. No. 4,899,747 discloses a tube of the modified “lavacuator®” type for introducing an inflatable intragastric balloon into the stomach of a patient. For this purpose, the distal end of the tube has a longitudinal slit. Eyelets are formed on either side of the slit, with a thread being passed in a “zigzag” configuration through the eyelets to close the cover. Several redundant loops of the thread are formed at the distal end of the tube so that an initial traction force on the free end of said thread enables the slit to be closed, while also allowing the thread to be withdrawn completely by applying a second traction force that is stronger than the first.

Such a closure system turns out to be unreliable in implementation for the following reasons.

Firstly, the slit is properly closed only by exerting a certain amount of tension on the thread, which tension needs to be maintained all along the path followed by the balloon from the mouth to the stomach. Such tension is therefore particularly difficult to maintain at a constant value since the path followed along the esophagus is relatively sinuous. In addition, in the event of too much tension being applied to the thread while it is being introduced could have the effect of undoing the terminal series of loops and thus releasing the slit, with all of the safety risks associated with such premature opening. Finally, even when constant closure tension can indeed be applied to the thread, which would appear to be extremely difficult if not impossible, it is still not certain that the terminal series of loops cannot come undone in untimely manner under the effect of the radially outward force that might be exerted by the folded balloon situated inside the tube, or indeed under the effect of the mechanical stresses to which the tube is bound to be subjected during preoperative handling, and also while it is being introduced into the stomach.

### SUMMARY OF THE INVENTION

Consequently, the objects given to the invention seek to remedy the various drawbacks mentioned above and to propose a novel kit and a novel cartridge for introducing an intragastric implant into the stomach of a patient in order to treat obesity, the kit and the cartridge enabling an implant to be positioned within the stomach in a manner that is particularly reliable and comfortable for the patient.

Another object of the invention is to propose a novel kit and a novel cartridge for introducing an intragastric implant into the stomach of a patient in order to treat obesity, which kit and cartridge are particularly simple and inexpensive in design.

Another object of the invention is to propose a novel kit and a novel cartridge for introducing an intragastric implant into the stomach of a patient in order to treat obesity, which kit and cartridge present small size and good dimensional uniformity and stability.

Another object of the invention is to propose a novel kit and a novel cartridge for introducing an intragastric implant into the stomach of a patient in order to treat obesity, which kit and cartridge are of construction that presents a good compromise between weight, strength, and packaging capacity.

Another object of the invention is to propose a novel kit and a novel cartridge for introducing an intragastric implant into the stomach of a patient in order to treat obesity, which kit and cartridge present non-traumatic contact for the tissue of the digestive organs.

Another object of the invention is to propose a novel kit and a novel cartridge for introducing an intragastric implant into the stomach of a patient in order to treat obesity, which kit and cartridge are particularly simple, effective, and repeatable to manufacture and use.

Another object of the invention is to propose a novel method of manufacturing a kit for introducing an intragastric implant into the stomach of a patient in order to treat obesity, which method is particularly simple to implement, while enabling a kit to be obtained that presents excellent reliability.

Another object of the invention is to propose a novel method of manufacturing a kit for introducing an intragastric implant into the stomach of a patient in order to treat obesity, which method enables a kit to be obtained that is small in size.

The objects given to the invention are achieved by a kit for introducing a surgical implant into a cavity in the body of a patient, the kit comprising:

a surgical implant for implanting in said cavity, said implant being expandable from a configuration for introduction into the cavity to a therapeutic configuration within the cavity; and

a cartridge for packaging said implant in the introduction configuration, said cartridge being provided with an opener member activatable by positive action enabling the cartridge to pass from a closed configuration in which it confines the implant in its introduction configuration, to an open configuration in which it allows said implant to expand;

the kit being characterized in that the cartridge includes locking means functionally connected to the opener member and capable on its own, without any external action on said locking means of holding the cartridge in the closed configuration.

The objects given to the invention are also achieved by a cartridge for introducing a surgical implant into a cavity within the body of a patient, said implant being designed to be implanted in said cavity and being expandable from a configuration for introduction into the cavity to a therapeutic configuration within the cavity, said cartridge being designed to package said implant in its introduction configuration and being provided with an opener member that is activatable by positive action enabling the cartridge to pass from a closed configuration in which it confines the implant in its introduction configuration, to an open configuration in which it allows said implant to expand, the cartridge being characterized in that it includes locking means functionally connected to the opener member and serving on its own to hold the cartridge in the closed configuration without any external action on said opener means.

The objects assigned to the invention are also achieved by a method of manufacturing a kit for introducing a surgical implant into a cavity within the body of a patient, the method comprising the steps of:

supplying or making a surgical implant for implanting in said cavity, said implant being expandable from a configuration for introduction into the cavity to a therapeutic configuration within the cavity;

supplying or making a cartridge for packaging said implant in the introduction configuration; and

providing said cartridge with an opener member activatable to enable the cartridge to pass from a closed configuration suitable for confining the implant in its introduction configuration, to an open configuration suitable for allowing said implant to expand;

the method being characterized in that it further comprises a step of locking the cartridge in the closed configuration, in which the cartridge is provided with locking means capable on its own, without any external action on said opener member, of holding the cartridge in the closed configuration, and in which said locking means is functionally connected to the opener member.

The objects assigned to the invention are also achieved by the use of a chain stitch in accordance with class 101 of the December 1982 standard NF G 05-002 as means for locking a cartridge for introducing a surgical implant into a cavity within the body of a patient.

### BRIEF DESCRIPTION OF THE DRAWINGS

Other objects and advantages of the invention appear better on reading the following description and from the accompanying drawings given purely by way of non-limiting illustration and in which:

FIG. 1 is a perspective view of a cartridge of the invention for introducing an intragastric implant into the stomach of a patient in order to treat obesity;

FIG. 2 is a side view of an implementation detail of the locking means of a cartridge constituting a preferred embodiment;

FIG. 3 is a view from beneath showing the implementation detail of FIG. 2;

FIG. 4 is a diagram showing the principle steps in the manufacturing method of the invention, in a first particular implementation thereof;

FIG. 5 is a perspective view showing a detail of a particular embodiment of an introduction cartridge of the invention; and

FIG. 6 is a diagram showing a second particular implementation of the method of the invention.

### BEST MANNER OF PERFORMING THE INVENTION

In the description below, reference is made purely by way of preferred example to a gastric implant for treating obesity, designed to be introduced within the stomach in order to reduce its capacity. Nevertheless, the invention is not limited to this preferred application, and on the contrary seeks to cover other surgical implants designed to be introduced into cavities within the body other than the gastric cavity, for example body cavities defined by an organ that forms a pouch or a duct.

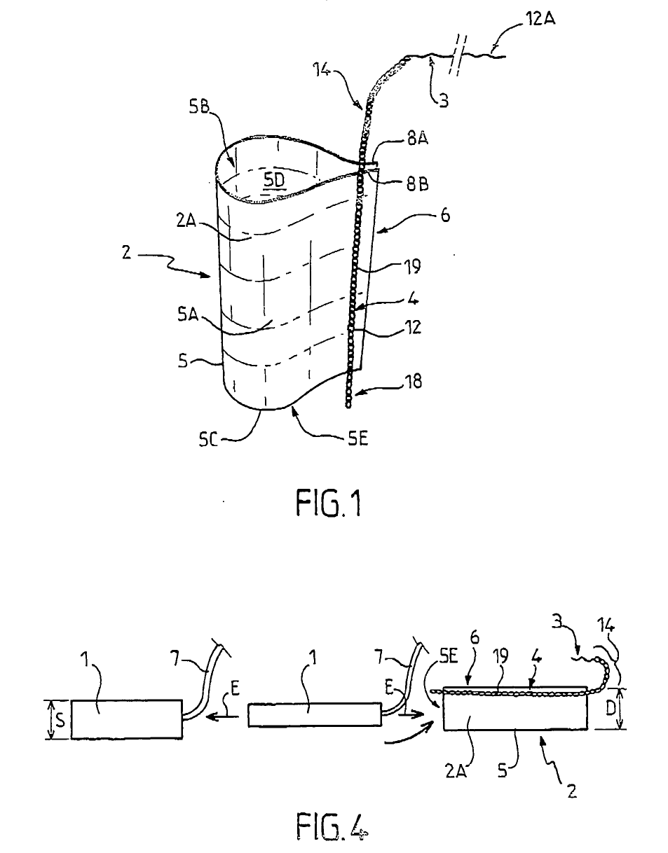

FIGS. 1 to 6 show a kit for introducing an intragastric implant 1 into the stomach of a patient in order to treat obesity, and also its implementation details.

Such a kit is intended to enable an implant 1 to be introduced into the stomach of a patient in order to reduce the volume of the stomach, insofar as said implant occupies a major fraction of the space available for food.

The kit of the invention is preferably intended to be suitable for being implanted via natural pathways, i.e. via the mouth and the esophagus, and preferably under endoscopic control (endoscopic implantation).

In accordance with the invention, the introducer kit comprises an intragastric implant 1 (shown diagrammatically in FIG. 4) that is to be implanted in the stomach in order to reduce its volume, said implant being expandable from a configuration for introduction into the stomach in which it presents a small volume and a shape suitable for passing along the natural pathways, and a therapeutic configuration within the stomach in which the implant occupies a predetermined volume, for example of the order of 600 milliliters (mL) corresponding to its volume in use.

Reference is made below to an implant 1 constituted by a balloon comprising at least one flexible bag that can be expanded by being filled with fluid, said bag being constituted by an envelope made using flexible materials, e.g. a silicone-type elastomer.

Nevertheless, it is entirely possible, without going beyond the ambit of the invention, to envisage the implant being formed by a structure that is not naturally flexible but rather is rigid or semi-rigid. In this respect, the implant could be constituted by a deployable structure that does not require fluid to be fed to it in order to be expanded, but that is expanded by an elastic effect or by making use of material having shape memory.

Preferably, the intragastric implant 1 is an intragastric balloon comprising a first flexible bag defining a predetermined inner volume, said first flexible bag being provided with first connection means having an orifice and a valve for receiving a connection member 7 of the catheter type, for connection to a first source of fluid (e.g. gas or liquid), in order to expand said first bag inside the stomach by filling the bag with said fluid.

In more preferred manner, the expandable intragastric balloon 1 of the invention includes at least one second flexible bag likewise of predetermined volume and provided with second connection means having an orifice and a valve, said second connection means possibly being separate and distinct from the first connection means so as to be capable of being connected to a second source of fluid.

By means of this disposition and with the two corresponding connection means being separate and independent, the two inside volumes of the bags are also made independent, thus making it possible to expand and inflate each of the bag using a different fluid, i.e. using fluids of different densities.

In preferred manner, said second bag is of smaller volume than the first and is located inside the first bag.

Consequently, for a given total overall volume of the intragastric balloon 1 presenting an external volume that is comparable to prior art bag devices, it is possible to obtain a lower weight for the two-bag intragastric balloon than can be obtained with prior art balloons.

Such a two-bag balloon is described in patent application PCT/FR 02/04589 in the name of the Applicant, and the content thereof is incorporated herein by reference.

The introducer kit of the invention also comprises a cartridge 2 for packaging said implant 1 while it is in its introduction configuration.

Preferably, the introduction configuration corresponds to the implant 1 being substantially tubular in shape, i.e. with the implant extending substantially along a single direction in three-dimensional space.

The cartridge 2 serves to contain and sheathe the implant 1 in its introduction configuration so as to co-operate with said implant in forming a streamlined assembly that is compact and of generally regular outside surface to make it easier to introduce via the mouth and the esophagus.

In accordance with the invention, the cartridge 2 is provided with an opener member 3 that can be activated by a positive action enabling it to go from a closed configuration (as shown in particular in FIGS. 1 and 4) in which it is suitable for confining the implant 1 in its introduction configuration, to an open configuration (not shown in the figures), in which it allows said implant 1 to expand, i.e. it releases the implant 1 from any interaction that prevents it from expanding.

The term “activatable by positive action” is used herein to mean that the opener member 3 can be activated on command, i.e. by the doctor performing introduction, in contrast to passive opener members, which might be activated by the balloon being expanded, for example (as happens with a pre-slit elastomer cover).

According to an important characteristic of the invention, the cartridge 2 has locking means 4 enabling the cartridge 2 to be held in its closed configuration without any external action on said means 4.

In other words, the locking means 4 lock the cartridge 2 in the closed configuration without any external action for maintaining said locking being required, for example no tensioning is required.

The term “locking” is used herein to mean more particularly that the cartridge 2 remains firmly in its closed configuration even when subjected to external forces tending to cause it to change towards its open configuration.

The locking means 4 is functionally connected to the opener member 3, such that when the opener member 3 is activated, it eliminates the effect of the locking means 4, thereby allowing the cartridge 2 to pass to its open configuration.

Thus, the cartridge 2 can pass from its closed configuration to its open configuration only once the opener member 3 has been activated, and remains insensitive to any other external stress that might be applied.

Thus, by structure, the cartridge 2 is of a closed configuration and it is naturally locked in its closed position.

Advantageously, the cartridge 2 comprises a sleeve 5 of substantially tubular shape. The sleeve 5 is defined by a side envelope 5A extending between a proximal end 5B and a distal end 5C. In the embodiment shown in FIG. 1, the sleeve has an axial opening 5E, 5D at each of its distal and proximal ends. Nevertheless, it is entirely possible, without going beyond the ambit of the invention, to envisage the sleeve 5 being closed at one of its ends, preferably its proximal end 5D, or even for it to be closed at both ends.

Advantageously, the proximal end 5B of the sleeve is assembled, e.g. by adhesive, to the connection member 7 that is connected to the implant 1, so that when the connection member 7 is withdrawn from the body of the patient after the implantation has been completed, it takes the cartridge 2 with it, leaving only the implant 1 inside the patient's stomach.

The sleeve 5 is provided with at least one side opening 6 formed over all or part of its length. As shown in FIG. 1, said side opening 6 can extend substantially longitudinally over the length of the sleeve 5. However it is entirely possible, without going beyond the ambit of the invention, for said side opening 6 to extend in any other way, for example transversely or helically. Said side opening 6 is closed by the locking means 4 when the cartridge 2 is in the closed configuration (FIG. 1), said side opening 6 being disengaged so as to allow the implant 1 to expand when the cartridge 2 is in its open configuration (not shown).

Preferably, the sleeve 5 is slit over all or part of its length, said slit constituting the side opening 6.

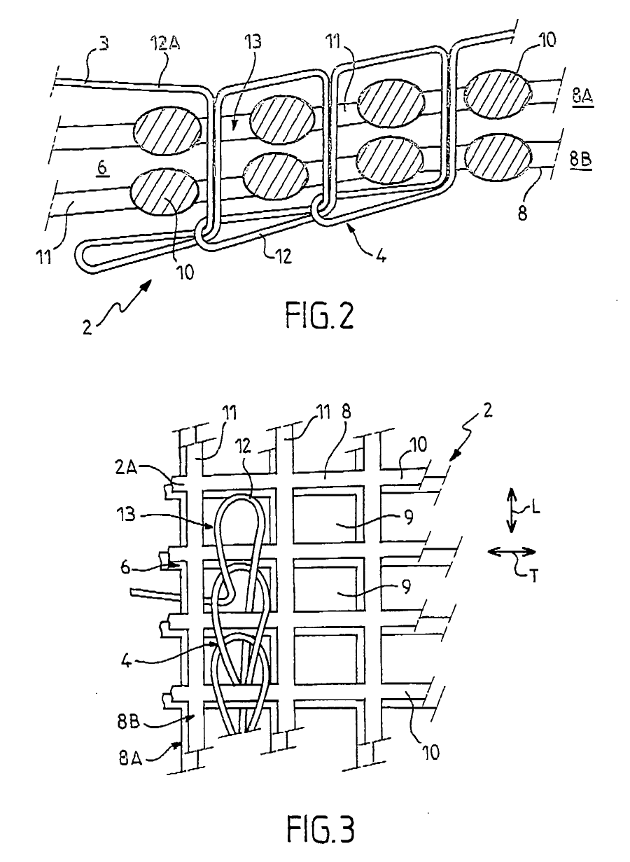

Advantageously, the sleeve 5 is constituted by a textile grid 8, as can be seen more particularly in FIGS. 2 and 3. The textile grid 8 comprises an array of crossed weft and warp threads 10 and 11 defining empty zones 9 so that the grid presents an open structure.

The grid 8 may be obtained by any method known to the person skilled in the art, for example it may optionally be woven, threaded, or even knitted.

Preferably, the array of weft and warp threads 10 and 11 is obtained with the weft and warp threads crossing at 90° (square structure), as shown in FIG. 3.

Advantageously, the sleeve 5 is constituted by a textile grid 8 having two opposite edges 8A and 8B interlocked by the locking means 4 in such a manner that the grid 8 becomes substantially tubular in shape.

In the embodiment shown in FIG. 1, the sleeve 5 is thus constituted by a textile grid 8 that is generally in the form of a rectangle having four sides that are parallel in pairs. The sleeve 5 is obtained by joining together two opposite sides.

Preferably, the textile grid 8 is made by knitting multi-strand polyester threads. Nevertheless, it is entirely possible to envisage using other types of thread, in particular single-strand polyester threads, single-strand or multi-strand polypropylene threads, or indeed threads of cotton, cellulose, or silk.

In general, it is particularly appropriate in the invention to use a grid 8 of the type used for making parietal reinforcing plates, such as those used for treating hernias or eventrations, for example.

Nevertheless, it is not essential to use a textile grid in the context of the invention, and it is possible to make the cartridge 2 from any other structure, for example from a continuous structure (not perforated) such as a fabric (preferably with a very tight weave and of small thickness) or using a membrane of plastics material.

In particular, in a preferred embodiment, the sleeve 5 is made of a fabric with two opposite edges that are interlocked by the locking means 4 in such a manner that the fabric is given a substantially tubular shape. The fabric is advantageously made by weaving threads that are based for the most part on polyamide, such as Nylon® threads.

By way of example, the sleeve 5 may be made by a substantially rectangular piece of commercially available parachute fabric, such as the fabric made using a “Rip stop” weave with threads of high-tenacity polyamide 6.6.

Preferably, the fabric used is subjected to cleaning and de-greasing operations so as to eliminate any possible toxicity.

Advantageously, the sleeve 5 is made from a material that is flexible but substantially not elastic. In other words, the material constituting the sleeve 5 is selected to present ability to be folded or rolled up, while nevertheless presenting a certain degree of longitudinal and transverse strength, like a sheet of paper.

Thus, when the sleeve is a textile grid 8 or a fabric, the grid or fabric presents little or no ability to extend in the longitudinal direction L or in the transverse direction T, but that does not prevent the grid 8 or the fabric presenting a certain amount of intrinsic flaccidity.

Under such circumstances, the sleeve 5 presents substantially no ability to stretch radially.

Advantageously, at least a portion of the surface 2A of the cartridge 2 is covered in a protective coating seeking to make the cartridge 2 easier to slide over an external surface.

Advantageously, the entire outside surface of the cartridge 2, i.e. the surface that is to come into contact with the esophagus, is covered in said protective coating.

Preferably, the entire inside surface of the cartridge 2, i.e. its surface that is to come into contact with the folded implant 1, is likewise covered in the protective coating.

Said coating serves to avoid any aggressive contact between the cartridge 2 and mouth or esophagus tissue, thus making it easier to pass the cartridge 2 all the way to the stomach.

Preferably, the coating is based on one or more materials taken from the following group:

a biocompatible elastomer, of the silicone or polyurethane type;

paraxylilene, of the parylene® type;

polyvinylpyrrolidone (PVP); and

sodium hyaluronate.

In other words, the coating serves essentially to improve the non-traumatic nature of the cartridge 2.

Naturally, without going beyond the ambit of the invention, it is possible to envisage the coating being constituted by any other biodegradable and biocompatible polymer.

Advantageously, the cartridge 2 is provided with a thread 12 having a first portion 19 constituting a fastening stitch, said stitch constituting the locking means 4, and a second portion 14, 12A that remains free so as to form the opener member 3 for actuating by applying traction.

The stitch constituting the first portion 19 is selected to be one that comes undone when sufficient traction is exerted on the free portion 14, 12A of the thread 12. Nevertheless, the stitch is also selected to be one that is stable on its own, producing intrinsic connection without there being any need to maintain tension or any other external action on the stitched portion 19.

Advantageously, the cartridge 2 has a thread 12 with a first portion 19 sewn with a single-thread chain stitch, so as to form the locking means 4, and a second portion 14, 12A that remains free and forms the opener member 3 that can be actuated in traction.

Chain stitch is a stitch that is well known per se and is obtained by looping the thread onto itself. In the context of the invention, the preferred stitch is the single-thread chain stitch of class 101 in the December 1982 standard NF G 05-002.

The invention makes use of the following very particular property of this stitch. This stitch, like other sewing stitches, serves to provide a locked assembly. Nevertheless, if traction is applied to the free end 12A of the single and continuous thread that has been used for sewing the stitch, and on this condition alone, then the stitch will become undone in cascade, thereby undoing the assembly and thus undoing the locking.

Chain stitch thus makes it possible using a single continuous thread to provide locking means 4 and an opener member 3 that are distinct, given that they require different actions for their respective activations.

Advantageously, the periphery of the side opening 6 in the sleeve 5 is provided with eyelets 13 for being assembled together by sewing with a single-thread chain stitch so as to close said side opening 6.

In the embodiment of the sleeve 5 using a textile grid 8 or a fabric, the eyelets 13 are defined by the mesh of the grid 8 (or the fabric) situated close to and along the two opposite edges 8A and 8B that are to be locked together. Under such circumstances, the chain stitches hold the weft threads 10 together in pairs along the opposite edges 8A and 8B, as shown in FIG. 2. The chain stitch is implemented so that it prevents the edges 8A and 8B moving relative to each other and prevents them from moving apart or becoming separated. Nevertheless, when traction is exerted on the end 12A of the thread 12, then the chain stitch becomes undone, thereby eliminating any closure connection between the edges 8A and 8B.

As shown in FIG. 2, the terminal end 12A corresponds to the free left-hand end of the thread 12 when the stitch is sewn from right to left.

Advantageously, and as shown in FIG. 1, the chain stitch is sewn not only through the grid 8 itself in order to make a cartridge 2 in the closed configuration, but also beyond the grid 8 and at both ends 14 and 18 in line with the stitching 19 implemented through the grid 8. This disposition makes it possible to guarantee reliably that the two edges 8A and 8B are locked together, in particular in the event of untimely traction being applied to the free terminal end 12A of the thread 12.

Preferably, and as shown in FIG. 5, the opener member 3 includes safety means 21 providing even more reliable means for preventing any untimely activation of the opener member 3.

When said opener member 3 is constituted by a terminal portion 14 of the thread 12 sewn with a chain stitch, the safety means 21 is advantageously constituted by a loop 20 interacting with two chain stitches 21 and 22 of said terminal portion 14, possibly two successive stitches, so as to prevent any running of the chain stitches in cascade situated downstream therefrom, i.e. beyond the two stitches 21, 22 and the sewn portion 19, when traction is applied to the terminal portion 12A.

Said loop 20 can be made using a length of thread, having its two ends knotted together at 22.

Said loop 20 is preferably located on the portion 14 forming the opener member 3 in such a manner as to be situated outside the body of the patient when the cartridge 2 is positioned in the stomach.

Thus, it suffices for the practitioner to undo the knot 22 in the loop 20 or merely to cut it, so as to allow the chain stitches to run undone in cascade when traction is exerted on the free end 12A.

The portions 14, 12A of the thread 12 forming the opener member 3 is dimensioned so as to be long enough to allow the practitioner to pull on the end 12A of the thread from the outside when the cartridge 2 is inside the patient's stomach, in order to open the cartridge 2 and release the implant 1. The portion 14 may preferably be introduced in a catheter, for example a catheter that is coaxial with or that coincides with the catheter 7 used for inflation.

The description above relates to a chain stitch interacting with eyelets 13. Naturally, the chain stitch could be made directly through a fabric that does not have openings or through a continuous membrane, for example, without thereby going beyond the ambit of the invention.

The thread(s) from which the locking means 4 is made can be manufactured from synthetic or natural materials, e.g. polyester, polypropylene, cotton, cellulose, silk, using any structure well known to the person skilled in the art (single- or multi-stranded, twisted, . . . ).

In an alternative embodiment of the locking means 4, instead of using chain-stitch sewing, it is possible to envisage implementing a miniaturized zip type closure system, with the moving member of the zip closure system being connected to a traction thread constituting the opener member.

In another alternative embodiment, the locking means 4 may be constituted by a Ziplock® type system with a slider, said slider enabling the cartridge 2 to be opened or closed, and being connected for this purpose to a traction thread constituting the opener member 3.

In a third alternative embodiment, the locking means 4 is constituted by a Velcro® type closure, the opener member 3 being formed by a thread arranged to exert a separation force on the complementary Velcro® elements when traction is exerted on said thread.

The invention also relates to a method of manufacturing a kit for introducing an intragastric implant 1 into the stomach of a patient in order to treat obesity, the method comprising the steps of:

supplying or making an intragastric implant 1 for implanting in the stomach in order to reduce its volume, said implant 1 being expandable from a configuration for introduction in the stomach to a therapeutic configuration within the stomach;

supplying or making a cartridge 2 for packaging said implant in the introduction configuration; and

providing said cartridge 2 with an opener member 3 activatable by positive action enabling the cartridge to pass from a closed configuration in which it is suitable for confining the implant 1 in its introduction configuration, to an open configuration in which it is suitable for allowing said implant 1 to expand.

In accordance with an important characteristic of the method of the invention, the method further comprises a step of locking the cartridge 2 in the closed configuration in which the cartridge 2 is provided with locking means 4 capable on its own, without requiring any external action on said means 4, of holding the cartridge 2 in the closed configuration, and in which said locking means 4 is functionally connected to the opener member 3.

Preferably, the cartridge 2 is made so that when it is in the closed configuration, it is substantially in the form of a sleeve with at least one axial opening 5D, 5E at one of the ends 5B, 5C of said sleeve 5.

Advantageously, the method in accordance with the invention includes a step of inserting the implant 1 into the sleeve 5, in which, and as shown in FIG. 4:

the implant 1 is shaped into its introduction configuration, such that said implant 1 presents a generally elongate shape of cross-section S;

then the implant 1 is prestressed so as to present a substantially elongate shape in which the cross-section S is reduced, so as to be smaller than the cross-section D of the sleeve 5;

then the prestressed implant 1 is introduced into the sleeve 5 through said at least one axial opening 5E; and

then, once the sleeve 5 contains the implant 1, the prestress is released such that the implant 1 returns to its introduction configuration.

For an intragastric balloon presenting an elastic nature, which applies to balloons made of elastomer material, the prestress may consist in exerting longitudinal traction on the balloon while in the introduction configuration (as represented by arrows E in FIG. 4). When stressed in this way, the balloon 1 is subjected to longitudinal stretching accompanied by constriction that reduces its cross-section S. While prestressed in this way the balloon can be inserted easily into the sleeve 5, even if the section D of the sleeve is smaller than the section S of the balloon 1 in its introduction configuration. Thereafter, the locking means 4 guarantees that the sleeve 5 does not open up under the effect of the radial centrifugal resilient return force exerted by the balloon 1 once the prestress has been removed.

Thus, the sleeve 5 can genuinely compress the balloon 1 so as to minimize the transverse size of the assembly formed by the sleeve 5 and the balloon 1.

This feature makes it possible to obtain sleeve 5 and balloon 1 assemblies of cross-section contained within a circle of diameter that is less than or equal to 18 millimeter (mm), even when the implant 1 is constituted by a balloon comprising two concentric bags.

In a variant implementation of the method of the invention, the step of inserting the implant 1 in the sleeve 5 comprises the following substeps:

shaping the implant 1 into the introduction configuration; and

then progressively stretching the implant 1 along its length using a jig 23 so as to reduce the cross-section S of said implant 1 while simultaneously and progressively covering the implant 1 with the sleeve 5 in its closed configuration.

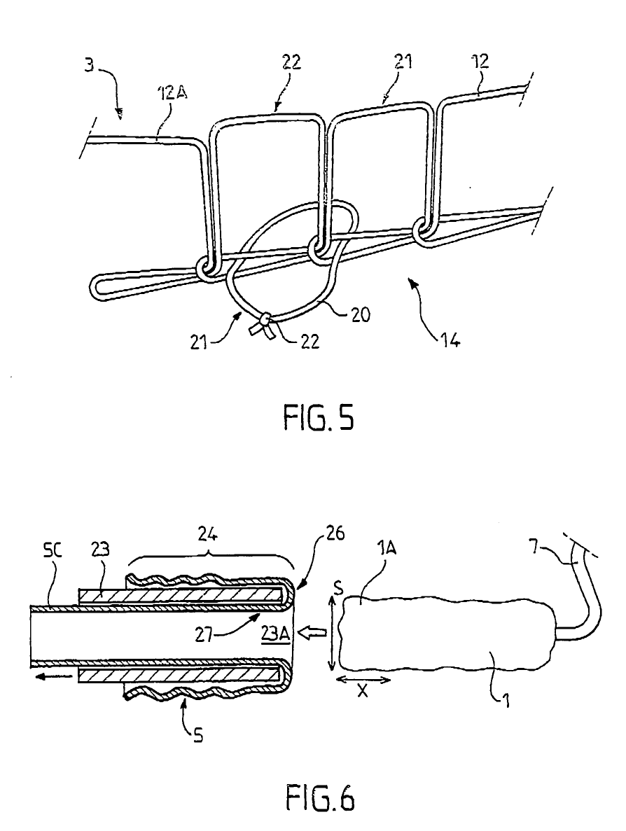

As shown in FIG. 6, the jig 23 preferably comprises a hollow cylindrical tube, preferably made of a material presenting a low coefficient of friction with the material constituting the sleeve 5. When the sleeve 5 is made of a textile material, for example a polyester grid, the jig 23 may advantageously be made of stainless steel.

The sleeve 5 in the closed configuration is engaged via it distal end 5C inside the hollow tube forming the jig 23, while the proximal portion 24 of the sleeve 5 that does not lie inside the hollow tube 23 is turned inside out so as to cover the outside of said tube 3.

This provides a hollow tube 3 whose inside and outside walls are covered and sheathed, at least in part, by the sleeve 5. One of the openings 23A of the hollow tube 23, referred to below as its inlet opening, corresponds to the opening where the cross-section of the hollow tube 23 is covered by the fold 26 formed by the sleeve 5 on the tube 23.

The implant 1 after it has been put into its introduction configuration is then inserted by force into the inlet opening 23A via its distal end 1A over a length X that is sufficient to establish friction contact between said proximal end 1A and the corresponding zone 27 of the sleeve 5.

Once this step of priming the implant 1 has been performed, it then suffices to exert traction on the distal end 5C of the sleeve 5 along the axis of the tube 23 so as to bring the implant 1 by friction towards the opening of the tube 23 that is opposite from its inlet opening 23A.

The implant 1 is thus moved without exerting any force directly on said end 1A, but merely by making use of the implant 1 being entrained by friction by the inside face of the sleeve 5.

Movement of the implant 1 along the inside of the hollow tube 23 thus enables the proximal portion 24 of the sleeve 5 that was initially folded over the outside surface of the hollow tube 23 to be unrolled onto said implant.

The implant is thus simultaneously compressed radially and covered without exerting any direct traction on the implant, thus serving to minimize any risk of the implant being damaged.

Finally, the invention also relates to using a chain stitch in accordance with class 101 of the December 1982 standard NF G 05-002 as locking means 4 for locking a cartridge 2 for introducing an intragastric implant 1 into the stomach of a patient to treat obesity.

The operation of the introducer kit of the invention is described below.

Initially, the surgeon has a kit in which the balloon 1 is packaged in a cartridge 2. The proximal end of the cartridge 2 is secured to a catheter 7 that is used both for causing the cartridge 2 to open inside the stomach and to inflate the balloon 1. The operation then takes place as follows.

The surgeon introduces the distal end 5C of the assembly constituted by the balloon 1 and the cartridge 2 into the patient's mouth. Under endoscopic control and by pushing on the catheter 7, the practitioner causes the kit to move along the esophagus towards the stomach until it reaches the stomach.

The practitioner then takes hold of the terminal end 12A of the thread 12 coming out of the proximal end of the catheter 7 and exerts traction on said thread 12 so as to undo the chain-stitch sewing 19 constituting the locking means 4. Once undone in this way, the sleeve 5 tends naturally to return to a flat shape, particularly if its has shape memory properties. Thereafter, the practitioner inflates the balloon 1 by delivering inflation fluid via the catheter 7. Once the balloon has been inflated, the practitioner jerks the catheter 7 so that, under the effect of its own weight, the balloon 1 separates from the catheter 7. Thereafter the practitioner withdraws the catheter 7 from the body of the patient, which catheter has the cartridge 2 attached thereto, e.g. by means of adhesive.

The inflated balloon thus floats freely in the stomach, and can perform its therapeutic function.

Finally, it should be observed that the cartridge 2 of the invention can be used for introducing any other expandable medical device into a body cavity, and that its use is not limited to implanting implants that are specifically intragastric implants.

The invention also relates to a novel method of surgical and therapeutic treatment implementing the kit, or merely the cartridge of the invention.

### SUSCEPTIBILITY OF INDUSTRIAL APPLICATION

The invention is applicable to implantable devices for treating obesity.

1. A kit for introducing a surgical implant into a cavity in the body of a patient, the kit comprising:
a surgical implant for implanting in said cavity, said implant being expandable from a configuration for introduction into the cavity to a therapeutic configuration within the cavity; and
a cartridge for packaging said implant in the introduction configuration, said cartridge being provided with an opener member activatable by positive action enabling the cartridge to pass from a closed configuration in which the cartridge confines the implant in the implant's introduction configuration, to an open configuration in which the cartridge allows said implant to expand;
wherein the cartridge includes locking means functionally connected to the opener member and capable on its own, without any external action on said locking means of holding the cartridge in the closed configuration.

2. The kit of claim 1, wherein the cartridge further comprises a sleeve provided with at least one side opening formed along its length, said opening being closed by said locking means when said cartridge is in the closed configuration, and said opening being disengaged to allow the implant to expand when said cartridge is in the open configuration.

3. The kit of claim 2, wherein the sleeve is substantially tubular in shape and is slit along all or part of its length, said slit constituting the side opening.

4. The kit according to claim 2, wherein the sleeve is made of a material that is flexible, but not substantially elastic.

5. The kit of claim 2, wherein the sleeve is made of a fabric having two opposite edges locked together by the locking means so that the fabric takes up a substantially tubular shape.

6. The kit of claim 5, wherein the fabric is made of woven polyamide threads.

7. The kit of claim 2, wherein at least a portion of the structure of the cartridge is covered in a coating for making the cartridge slide more easily against an external surface.

8. The kit of claim 7, wherein the coating includes a material selected from the group consisting of:
a biocompatible elastomer, of the silicone or polyurethane type;
paraxylilene;
polyvinylpyrrolidone (PVP); and
sodium hyaluronate.

9. The kit claim 1, wherein the cartridge is provided with a thread having a first portion sewn as a single-thread chain stitch so as to form said locking means, and having a second portion that remains free and forms the opener member that can be actuated in traction.

10. The kit of claim 9, wherein, the periphery of the side opening is provided with eyelets for being assembled together by single-thread chain-stitch sewing in order to close said opening.

11. The kit of claim 10, wherein the eyelets are defined by meshes in the fabric situated close to and along said edges.

12. (canceled)

13. A kit for introducing an intragastric implant into a stomach of a patient to treat obesity, the kit comprising:
an intragastric implant for implanting in the stomach in order to reduce its volume, said implant being expandable from a configuration for introduction into the stomach to a therapeutic configuration within the stomach; and
a cartridge for packaging said implant in the introduction configuration, said cartridge being provided with an opener member that is activatable by positive action, enabling the cartridge to pass from a closed configuration in which the cartridge confines the implant in the implant's introduction configuration, to an open configuration in which the cartridge allows said implant to expand, the cartridge including locking means functionally connected to the opener member and capable on its own, without requiring any external action on said locking means, of holding the cartridge in the closed configuration.

14. The kit of claim 13, wherein the intragastric implant is an intragastric balloon comprising a first flexible bag defining a predetermined inside volume, said first flexible bag being provided with first connection means for receiving a connection member for connection to a first source of a fluid in order to enable said first bag to be expanded in the stomach by being filled with the fluid.

15. The kit of claim 14, wherein the balloon includes at least one second flexible bag of predetermined volume and provided with second connection means so as to enable it to be connected to a second source of fluid.

16. The kit of claim 15, wherein said at least one second flexible bag is of smaller volume than the first flexible bag, and is located inside the first flexible bag.

17. A cartridge for introducing a surgical implant into a cavity within the body of a patient, said implant (being designed to be implanted in said cavity and being expandable from a configuration for introduction into the cavity to a therapeutic configuration within the cavity, said cartridge being designed to package said surgical implant in the surgical implant's introduction configuration and being provided with an opener member that is activatable by positive action enabling the cartridge to pass from a closed configuration in which the cartridge confines the surgical implant in the surgical implant's introduction configuration, to an open configuration in which the cartridge allows said surgical implant to expand, wherein the cartridge includes locking means functionally connected to the opener member and serving on its own to hold the cartridge in the closed configuration without any external action on said opener means.

18. The cartridge of claim 17, wherein the cartridge further comprises a sleeve provided with at least one side opening formed in its length, said side opening being closed by said locking means when said cartridge is in the closed configuration, and said opening being disengaged to allow the surgical implant to expand when said cartridge is in the open configuration.

19. The cartridge of claim 18, wherein the sleeve is substantially tubular in shape and is slit along at least a part of its length, said slit constituting said side opening.

20. The cartridge of claim 18 wherein, the sleeve is made of a material that is flexible, but not substantially elastic.

21. The cartridge of claim 18, wherein the sleeve is made of a fabric having two opposite edges locked together by the locking means so that the fabric takes up a substantially tubular shape.

22. The cartridge of claim 21, wherein the fabric is made of woven polyamide threads.

23. The cartridge according to claim 18, wherein at least a portion of its surface is covered in a coating for making the cartridge slide more easily against an external surface.

24. The cartridge according to claim 23, wherein the coating includes a material selected from the group consisting of:
a biocompatible elastomer, of the silicone or polyurethane type;
paraxylilene,
polyvinylpyrrolidone (PVP); and
sodium hyaluronate.

25. The cartridge of claim 17, wherein the cartridge further includes a thread having a first portion sewn with a single-thread chain stitch so as to form the locking means, and having a second portion that remains free and forms the opener member that can be actuated in traction.

26. The cartridge of claim 25, wherein the periphery of the side opening is provided with eyelets for being assembled by sewing with a single-thread chain stitch in order to close said opening.

27. The cartridge of claim 26, wherein the sleeve is made of a fabric having two opposite edges locked together by the locking means so that the fabric takes a substantially tubular shape;
wherein the eyelets are formed by meshes in the fabric situated close to and along said edges.

28. (canceled)

29. A cartridge for introducing an intragastric implant into the stomach of a patient in order to treat obesity, said implant being designed to be implanted in the stomach in order to reduce its volume and being expandable from a configuration for introduction into the stomach to a therapeutic configuration within the stomach, said cartridge being designed to package said intragastric implant in the introduction configuration and being provided with an opener member activatable by positive action enabling the cartridge to pass from a closed configuration in which the cartridge confines the intragastric implant in the intragastric implant's introduction configuration, to an open configuration in which the cartridge allows said intragastric implant to expand, said cartridge including locking means functionally connected to the opener member and capable on its own, without any external action on said opener means, of holding the cartridge in the closed configuration.

30. A method of manufacturing a kit for introducing a surgical implant into a cavity within the body of a patient, the method comprising the steps of:
supplying or making a surgical implant for implanting in said cavity, said implant being expandable from a configuration for introduction into the cavity to a therapeutic configuration within the cavity;
supplying or making a cartridge for packaging said surgical implant in the introduction configuration; and
providing said cartridge with an opener member activatable to enable the cartridge to pass from a closed configuration suitable for confining the surgical implant in surgical implant's introduction configuration, to an open configuration suitable for allowing said surgical implant to expand; and
locking the cartridge in the closed configuration, in which the cartridge is provided with locking means capable on its own, without any external action on said opener member, of holding the cartridge in the closed configuration, and in which said locking means is functionally connected to the opener member.

31. The method of claim 30, wherein the step of locking the cartridge in the closed configuration further comprises locking the cartridge in substantially the shape of a sleeve, the sleeve including at least one axial opening at one of the ends of said sleeve.

32. The method of claim 31, further comprising the step of inserting the surgical implant in the sleeve by:
shaping the surgical implant into its introduction configuration; and
constrained constraining the surgical implant progressively along its length by means of a jig to reduce the cross-section of said surgical implant while simultaneously covering the surgical implant in the sleeve in the closed configuration.

33. A method of manufacturing a kit for introducing an intragastric implant into a stomach of a patient to treat obesity, the method including the steps of:
supplying or making an intragastric implant for implanting in the stomach to reduce the volume of the stomach, said implant being expandable from a configuration for introduction in the stomach to a therapeutic configuration within the stomach;
supplying or making a cartridge for packaging said implant in the introduction configuration; and
providing said cartridge with an opener member activatable by positive action enabling the cartridge to pass from a closed configuration in which the cartridge is suitable for confining the implant in the surgical implant's introduction configuration, to an open configuration in which the cartridge is suitable for allowing said implant to expand; and
locking the cartridge in the closed configuration in which the cartridge is provided with locking means capable on its own, without requiring any external action on said locking means, of holding the cartridge in the closed configuration, and in which said locking means is functionally connected to the opener member.

34. (canceled)
